(19) United States
(12) Patent Application Publication
(10) Pub. No.: US 2004/0043419 A1
(43) Pub. Date: Mar. 4, 2004

(54) Javelinization of protein antigens to heat shock proteins

(76) Inventors: James E. Rothman, New York, NY (US); Mee H. Hoe, Irvington, NY (US); Mark Mayhew, New York, NY (US)

Correspondence Address: KENYON & KENYON, ONE BROADWAY, NEW YORK, NY, 10004, US

(21) Appl. No.: 10/258,147

(22) PCT Filed: Apr. 17, 2001
(86) PCT No.: PCT/US01/12567

(51) Int. Cl. G01N 33/53; C07K 14/47

(52) U.S. Cl. 435/7.1; 530/403

(57) ABSTRACT

The present invention relates to antigenic complexes, wherein an antigenic complex comprises a peptide or protein containing a plurality of epitopes non-covalently joined to a heat shock protein via a molecular tether referred to as a “javelin”. Such complexes do not require that each epitope Be defined, and may in certain embodiments, elicit both antibody and cell-mediated immune reactions. The complexes of the invention may be used to induce therapeutic immune responses directed toward the treatment or prevention of infectious diseases and malignancies.

### 1. INTRODUCTION

The present invention relates to antigenic complexes, wherein an antigenic complex comprises a peptide or protein containing a plurality of epitopes non-covalently joined to a heat shock protein via a molecular tether referred to as a “javelin”. Such complexes do not require that each epitope be defined, and may, in certain embodiments, elicit both antibody and cell-mediated immune reactions. The complexes of the invention may be used to induce therapeutic immune responses directed toward the treatment or prevention of infectious diseases and malignancies.

### 2. BACKGROUND OF THE INVENTION

Heat shock proteins (“hsps”) constitute a highly conserved class of proteins selectively induced in cells under stressful conditions, such as sudden increases in temperature or glucose deprivation. Able to bind to a wide variety of other proteins in their non-native state, heat shock proteins participate in the genesis of these bound proteins, including their synthesis, folding, assembly, disassembly and translocation (Freeman and Morimoto, 1996, EMBO J. 15:2969-2979; Lindquist and Craig, 1988, Annu. Rev. Genet. 22:631-677; Hendrick and Hartl, 1993, Annu. Rev. Biochem. 62:349-384). Because they guide other proteins through the biosynthetic pathway, heat shock proteins are said to function as “molecular chaperones” (Frydman et al., 1994, Nature 381: 111-117; Hendrick and Hartl, Annu. Rev. Biochem. 62:349-384; Hartl, 1996, Nature 381:571-580). Induction during stress is consistent with their chaperone function; for example, dnak, the Escherichia coli hsp70 homolog, is able to reactivate heat-inactivated RNA polymerase (Ziemienowicz et al., 1993, J. Biol. Chem. 268:25425-25341).

The heat shock protein gp96 resides in the endoplasmic reticulum, targeted there by an amino-terminal signal sequence and retained by a carboxy-terminal KDEL amino acid motif (Lys-Asp-Glu-Leu (SEQ ID NO: 1); referred to hereafter as the “KDEL” sequence, which promotes endoplasmic reticulum recapture; Srivastava et al., 1987, Proc. Natl. Acad. Sci. U.S.A. 84:3807-3811). Found in higher eukaryotes but not in Drosophila or yeast, gp96 appears to have evolved relatively recently, perhaps by a duplication of the gene encoding the cytosolic heat shock protein hsp90, to which it is highly related (Li and Srivastava, 1993, EMBO J. 12:3143-3151; identity between human hsp90 and murine gp96 is about 48 percent). It has been proposed that gp96 may assist in the assembly of multi-subunit proteins in the endoplasmic reticulum (Wiech et al., 1992, Nature 358:169-170). Indeed, gp96 has been observed to associate with unassembled immunoglobulin chains, major histocompatability class II molecules, and a mutant glycoprotein B from Herpes simplex virus (Melnick et al., 1992, J. Biol. Chem. 267:21303-21306; Melnick et al., 1994, Nature 370:373-375; Schaiff et al., 1992, J. Exp. Med. 176:657-666; Ramakrishnan et al., 1995, DNA and Cell Biol. 14:373-384). Further, expression of gp96 is induced by conditions which result in the accumulation of unfolded proteins in the endoplasmic reticulum (Kozutsumi et al., 1988, Nature 332:462-464). It has been reported that gp96 appears to have ATPase activity (Li and Srivastava, 1993, EMBO J. 12:3143-3151), but this observation has been questioned (Wearsch and Nicchitta, 1997, J. Biol. Chem. 272:5152-5156).

Unlike gp96, hsp90 lacks the signal peptide and KDEL sequence associated with localization in the endoplasmic reticulum, residing, instead, in the cytosol. Although hsp90 has not been detected as a component of the translational machinery (Frydmann et al., 1994, Nature 370:111-116), it has been reported to be highly effective in converting a denatured protein, in the absence of nucleotides such as ATP or ADP, to a “folding competent” state which can subsequently be refolded upon addition of hsp70, hdj-1 and nucleotide (Freeman and Morimoto, 1996, EMBO J. 15:2969-2979; Schneider et al., 1996, Proc. Natl. Acad. Sci. U.S.A. 93:14536-14541). Hsp90 has been observed to serve as a chaperone to a number of biologically highly relevant proteins, including steroid aporeceptors, tubulin, oncogenic tyrosine kinases, and cellular serine-threonine kinases. (Rose et al., 1987, Biochemistry 26:6583-6587; Sanchez et al., 1988, Mol. Endocrinol. 2:756-760; Miyata and Yahara, 1992, J. Biol. Chem. 267:7042-7047; Doyle and Bishop, 1993, Genes Dev. 7:633-638; Smith and Toft, 1993, Mol. Endocrinol. 7:4-11; Xu and Lindquist, 1993, Proc. Natl. Acad. Sci. U.S.A. 90:7074-7078; Stancato et al., 1993, J. Biol. Chem. 268: 21711-21716; Cuttforth and Rubin, 1994, Cell 77:1027-1035; Pratt and Welsh, 1994, Semin. Cell Biol. 5:83-93; Wartmann and Davis, 1994, J. Biol. Chem. 269:6695-6701; Nathan and Lindquist, 1995, Mol. Cell. Biol. 15:3917-3925; Redmond et al., 1989, Eur. J. Cell. Biol. 50:66-75). Hsp90 has been observed to function in concert with other proteins, some of which may act as true chaperones, others serving only as accessories; for example, cellular assembly of the progesterone receptor has been reported to involve hsp90 and seven other proteins (Smith et al., 1995, Mol. Cell. Biol. 15:6804-6812).

Inoculation with heat shock protein prepared from tumors of experimental animals has been shown to induce immune responses in a tumor-specific manner; that is to say, heat shock protein gp96 purified from a particular tumor could induce an immune response which would inhibit the growth of cells from the identical tumor of origin, but not other tumors, regardless of relatedness (Srivastava and Maki, 1991, Curr. Topics Microbiol. 167:109-123). The source of the tumor-specific immunogenicity has not been confirmed. Genes encoding heat shock proteins have not been found to exhibit tumor-specific DNA polymorphism (Srivastava and Udono, 1994, Curr. Opin. Immunol. 6:728-732). High-resolution gel electrophoresis has indicated that tumor-derived gp96 may be heterogeneous at the molecular level; evidence suggests that the source of this heterogeneity may be populations of small peptides adherent to the heat shock protein, which may number in the hundreds (Feldweg and Srivastava, 1995, Int. J. Cancer 63:310-314). Indeed, an antigenic peptide of vesicular stomatitis virus has been shown to associate with gp96 in virus infected cells (Nieland et al., 1996, Proc. Natl. Acad. Sci. U.S.A. 93:6135-6139). It has been suggested that this accumulation of peptides is related to the localization of gp96 in the endoplasmic reticulum, where it may act as a peptide acceptor and accessory to peptide loading of major histocompatability complex class I molecules (Li and Srivastava, 1993, EMBO J. 12:3143-3151; Suto and Srivastava, 1995, Science 269:1585-1588).

Heat shock proteins have been used as adjuvants to stimulate an immune response (see, for example, Edgington, 1995, Bio/Technol. 13:1442-1444; PCT Application International Publication Number WO 94/29459 by the Whitehead Institute for Biomedical Research, Richard Young, inventor, and references infra). One of the best known adjuvants, Freund's complete adjuvant, contains a mixture of heat shock proteins derived from mycobacteria (the genus of the bacterium which causes tuberculosis); Freund's complete adjuvant has been used for years to boost the immune response to non-mycobacterial antigens. A number of references suggest, inter alia, the use of isolated mycobacterial heat shock proteins for a similar purpose, including vaccination against tuberculosis itself (Lukacs et al., 1993, J. Exp. Med. 178:343-348; Lowrie et al., 1994, Vaccine 12:1537-1540; Silva and Lowrie, 1994, Immunology 82:244-248; Lowrie et al., 1995, J. Cell. Biochem. Suppl. 0(19b):220; Retzlaff et al., 1994, Infect. Immun. 62:5689-5693; PCT Application International Publication No. WO 94/11513 by the Medical Research Council, Colston et al., inventors; PCT Application International Publication No. WO 93/1771 by Biocine Sclavo Spa, Rappuoli et al., inventors).

Other references focus on the ability of heat shock proteins to naturally form associations with antigenic peptides, rather than the classical adjuvant activity (see, for example PCT Application No. PCT/US96/13233 by Sloan-Kettering Institute for Cancer Research, Rothman et al., inventors; Blachere and Srivastava, 1995, Seminars in Cancer Biology 6:349-355; PCT Application International Publication No. WO 95/24923 by Mount Sinai School of Medicine of the City University of New York, Srivastava et al., inventors). In one protocol used by Srivastava in a phase I European clinical trial, cells prepared from a surgically resected tumor were used to prepare gp96, which was then reinoculated into the same patient (Edgington, 1995, Bio/Technol. 13:1442-1444). The fact that a new gp96 preparation must be made for each patient is a significant disadvantage. PCT Application International Publication No. WO 95/24923 (supra) suggests that peptides in heat shock protein complexes may be isolated and then re-incorporated into heat shock protein complexes in vitro. There is no evidence that this time-consuming procedure would be successful beyond the treatment of the patient from which the heat shock protein was derived. Further, the preparation of an effective quantity of heat shock protein requires the harvest, from the patient, of an amount of tissue which not every patient would be able to provide. Moreover, this approach limits the use of heat shock proteins as peptide carriers to those peptides with which a natural association is formed in vivo, and the affinity of such peptides for heat shock protein may be inadequate to produce a desired immune response using complexes generated in vitro.

Although the immunogenic potential of heat shock proteins and molecular chaperones has been clearly demonstrated (reviewed by Schild H. et al., Current Opinion in Immunology (1999) 11:109-113), whereby hsps are believed to deliver bound antigens to antigen presenting cells for subsequent display on MHC class I or class II molecules (thereby generating a T cell response), many antigens do not bind sufficiently well to heat shock proteins or molecular chaperones for them to be efficiently delivered.

In attempts to circumvent this limitation, heat shock proteins have been covalently joined to antigenic peptides of choice. For example, it has been reported that a synthetic peptide comprising multiple iterations of NANP (Asn Ala Asn Pro; SEQ ID NO: 2) malarial antigen, chemically crosslinked to glutaraldehyde-fixed mycobacterial heat shock proteins hsp65 or hsp70, was capable of inducing a humoral (antibody based) immune response in mice in the absence of further adjuvant; a similar effect was observed using heat shock protein from the bacterium Escherichia coli (Del Guidice, 1994, Experientia 50:1061-1066; Barrios et al., 1994, Clin. Exp. Immunol. 98:224-228; Barrios et al., 1992, Eur. J. Immunol. 22:1365-1372). Cross-linking of synthetic peptide to heat shock protein and possibly glutaraldehyde fixation were required for antibody induction (Barrios et al., 1994, Clin. Exp. Immunol. 98:229-233), and cellular immunity does not appear to be induced. In another example, Young et al., in PCT Application International Publication Number WO 94/29459, discloses fusion proteins in which an antigenic protein is joined to a heat shock protein.

A potential disadvantage of such covalent linkage approaches is that they tend to favor an antibody-based, rather than a cellular, immune response. In such context, the heat shock protein may act as a carrier to promote antibody responses to covalently linked proteins or peptides, a well known adjuvant function of immunogenic proteins. Furthermore, heat shock protein and antigen are irreversibly linked; this may alter the solubility of either protein component, or may create structural distortion which interferes with the association between antigen and critical major histocompatability complex components.

As an alternate approach to covalent linkage, antigenic proteins have been non-covalently bound to heat shock protein via a molecular tether which binds to heat shock protein under physiologic conditions. This tether is referred to as a “javelin” herein, and the process of complexing an antigenic protein or peptide with a heat shock protein is referred to as “javelinization”.

An example of the usefulness of “javelinization” is as follows. Heat shock protein 70 has been shown to be effective at delivering bound peptide antigens to antigen presenting cells for their display on MHC class I molecules. Immunization of mice with hsp70 bound antigens has resulted in the generation of strong cellular immune responses against the chosen antigen. However, in vitro experimentation has shown that many optimal MHC class I binding antigens, do not bind well to hsp70. In an attempt to optimize the binding of such antigenic peptides to hsp70, hybrid peptides engineered to contain an optimized hsp70 binding peptide (a “javelin” having a sequence Hy-x-Hy-x-Hy-x-Hy, where Hy corresponds to a hydrophobic amino acid and x corresponds to any amino acid, and more specifically His Trp Asp Phe Ala Trp Pro Trp; SEQ ID NO: 3), a linker (having the sequence GSG) and the antigenic peptide of choice, were synthesized. Such peptides bound well to hsp70 and when mice were immunized with these peptides bound to hsp70, the cellular immune responses generated were significantly stronger than those obtained from immunizations with un-javelinized versions of the antigenic peptides bound to hsp70 (International Patent Application No. PCT/US 96/13363, and Moroi Y. et al., (2000) Proc. Natl. Acad. Sci. 97:3485-90). From a product development point of view, however, the javelinization of short peptide antigens may be limited by the knowledge of which specific antigens to use. Further, it may be desirable, for therapeutic purposes, to produce an immune response toward more than one antigenic peptide, either in order to induce immunity of sufficient magnitude to eliminate a diseased cell or pathogen, or because different individuals may, by virtue of their major histocompatibility phenotype, be more or less responsive to particular antigens.

### 3. SUMMARY OF THE INVENTION

The present invention provides for an antigenic complex comprising a plurality of epitopes non-covalently bound to a heat shock protein via a javelin sequence. The plurality of epitopes are covalently bound to the javelin, optionally via a linker sequence. In certain embodiments, the epitopes are comprised in a larger protein, and may occur naturally in the context of said protein. As such, the characterization of particular epitopes within the protein is not required to practice the invention. In alternative embodiments, epitopes which may not occur together in nature are bound to a single javelin, optionally via a linker sequence, thereby providing a cocktail of antigens which, when non-covalently associated with heat shock protein in an antigenic complex, may be used to produce a therapeutic immune response in a subject.

Accordingly, the present invention overcomes a number of the limitations of the prior art. For example, specific knowledge of the T cell epitope (MHC class I binding peptide) is not required, and a specific product is not restricted to patients with a certain HLA haplotype as a larger protein will contain many T cell epitopes that can bind to the various HLA types. Furthermore, the same protein may contain MHC class II epitopes that can also be processed by antigen presenting cells to generate a helper T cell response as well as an antibody response. Yet another advantage of the present invention is that it may be used to identify antigens restricted to either MHC class I or class II, in that a javelin may deliver a protein to a cell, resulting in the binding of peptide to MHC components, and the bound peptide(s) may be eluted and sequenced.

### 4. BRIEF DESCRIPTION OF THE FIGURES

FIGS. 1A-D. Various embodiments of the invention. A. A protein comprising a plurality of epitopes (represented by a square, triangle, and circle) is covalently joined to a javelin molecule by a chemical linker (L). B. A plurality of epitopes originating from the same protein, comprised in isolated peptides, are covalently joined to a javelin by chemical linkers (L). C. A plurality of proteins (designated as an open shape and a cross-hatched shape, respectively) comprising a plurality of epitopes (represented by an open square, an open triangle, an open circle, a cross-hatched diamond, and a cross-hatched doughnut), are covalently joined to a javelin molecule by a chemical linker (L). D. A plurality of epitopes, as designated in (C), comprised in isolated peptides, are covalently joined to a javelin by chemical linkers. Where epitopes are comprised in isolated peptides, the peptides are designated by wavy lines.

FIG. 2. The protein sequence of ovalbumin (SEQ ID NO: 4). The bolded amino acids correspond to those of the domain 200-291. The major histocompatibility complex (“MHC”) class I epitope, SIINFEKL(SEQ ID NO: 5), is underlined and the MHC class II epitope, TEWTSSNVMEERKIKV(SEQ ID NO: 6), is double-underlined.

FIG. 3. The structure of ovalbumin. Ova200-291 is shown in blue and approximately circled, and SIINFEKL (SEQ ID NO: 5)is shown in red and approximately enclosed in a box.

FIG. 4. The nucleotide sequence of the ovalbumin cDNA(SEQ ID NO: 7). The ATG start codon is bolded as is the termination codon. Underlined are the 5′ and 3′ regions of the sequence that code for the OVA 200-291 domain.

FIGS. 5A-D. Tumor growth curves for mice immunized with TiterMax and buffer (group A), TiterMax and SIINFEKL peptide (SEQ ID NO: 5; group B), Javelin-Ova200-291-Javelin alone (group C), and Javelin-Ova200-291-Javelin bound to mouse hsp70 (group D).

### 5. DETAILED DESCRIPTION OF THE INVENTION

The present invention provides for an antigenic complex comprising a plurality of epitopes non-covalently bound to a heat shock protein via a javelin sequence. The plurality of epitopes are covalently bound to the javelin, optionally via a linker sequence.

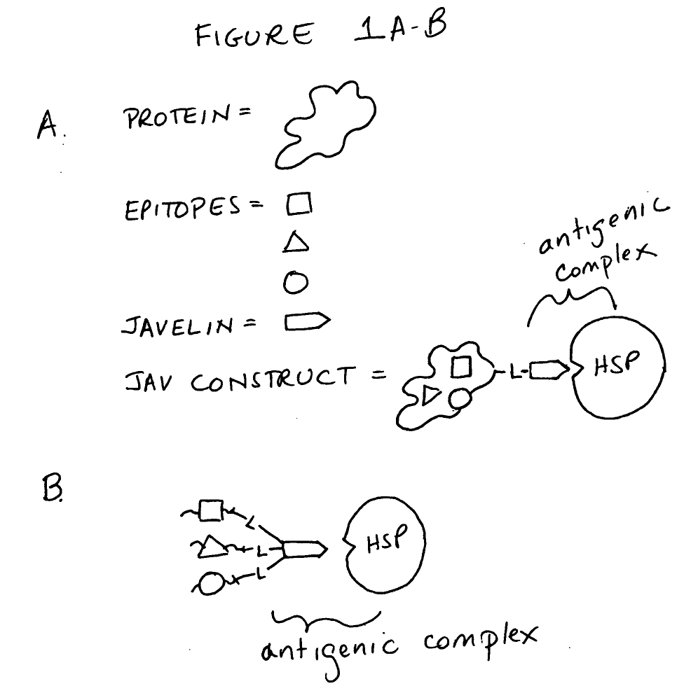

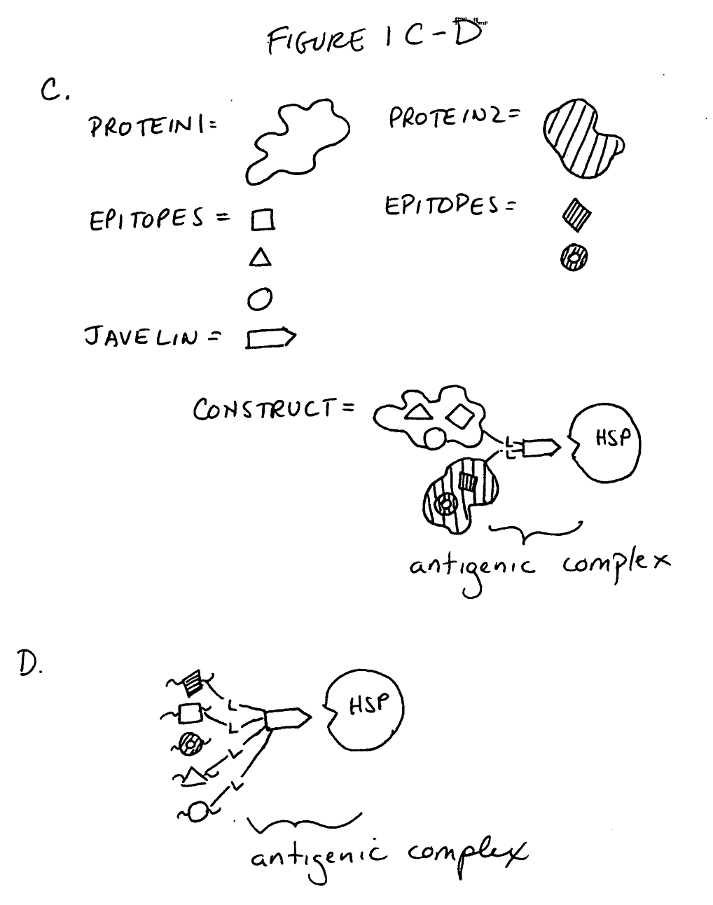

In a first set of embodiments (FIG. 1A and FIG. 1B), the present invention provides for an antigenic complex comprising a plurality of epitopes, non-covalently joined to a heat shock protein by a tethering molecule (“javelin”) having affinity for the heat shock protein, wherein the epitopes are covalently joined to the tethering peptide and wherein the epitopes are derived from a single antigenic protein. The epitopes may be comprised in the protein (FIG. 1A) or may be comprised in isolated peptides (FIG. 1B), and joined to the javelin via a chemical linker, which may be a covalent bond or may comprise one or more atoms (e.g. a peptide bond or a peptide linker).

In a second set of embodiments, the present invention provides for an antigenic complex comprising a plurality of epitopes, non-covalently joined to a heat shock protein by a tethering molecule (“javelin”) having affinity for the heat shock protein, wherein the epitopes are covalently joined to the tethering peptide and wherein the epitopes are derived from more than one antigenic protein. The epitopes may be comprised in the proteins (FIG. 1C) or may be comprised in isolated peptides (FIG. 1D), and joined to the javelin via a chemical linker, which may be a covalent bond or may comprise one or more atoms (e.g. a peptide bond or a peptide linker).

In a third set of embodiments, the present invention provides for an antigenic complex comprising a plurality of epitopes, non-covalently joined to a heat shock protein by a tethering molecule (“javelin”) having affinity for the heat shock protein under physiologic conditions, wherein the epitopes are covalently joined to the tethering peptide and wherein one epitope is a Class I epitope and the other epitope is a Class II epitope. For example, in FIG. 1A and FIG. 1B, the open square could represent a Class I epitope and the open circle could be a Class II epitope, and in FIG. 1C and FIG. 1D, the open square could represent a Class I epitope and the cross-hatched doughnut could represent a Class II epitope (it should be understood, however, that the MHC restrictions of the representative epitopes are only specified by way of explanation for this third set of embodiments and do not necessarily apply to all embodiments).

In a fourth set of embodiments, the present invention provides for one or more epitope, as comprised in an antigenic peptide, non-covalently joined to a heat shock protein by a plurality of tethering molecules (“javelins”), wherein the epitope or epitopes are covalently joined to the tethering molecule. The javelins may have the same or different chemcial structures (e.g. may have different peptide sequences).

As used herein, the term “heat shock protein” or “hsp” refers to any protein that has the capability to bind peptides or proteins and whose intracellular concentration increases (i.e., is “inducible”) when the cell is stressed, including non-inducible homologs of such proteins. Examples of heat shock proteins include but are not limited to gp96(grp94), hsp90, BiP, hsp70, hsp60, hsp40, hsc70, calnexin, calreticulin and hs10. Heat shock protein for use according to the invention may be prepared from a natural source, expressed recombinantly, or chemically synthesized.

The term “javelin” as used herein refers to a peptide or non-peptide sequence which non-covalently binds to heat shock protein under physiologic conditions. For example, but not by way of limitation, physiological conditions would include temperatures of 4-55° C., and preferably 20-40° C.; a pH of 3-12, and preferably 5-8; and ionic strengths approximating the ionic strength of 50-300 mM NaCl, and preferably 100-200 mM NaCl. A specific, nonlimiting example of physiologic conditions includes phosphate buffered saline (13 M NaH₂PO₄, 137 mM NaCl, pH 7.4) at 37° C. Such javelins may have amino acid compositions which comprise a substantial proportion of hydrophobic amino acids such as phenylalanine and tryptophan, and/or a substantial number of serine, threonine, or proline residues. In particular, nonlimiting embodiments, javelins of the invention may comprise amino acid sequences which have the general description hydrophobic-x-hydrophobic-x-hydrophobic-x-hydrophobic, where “hydrophobic” denotes a hydrophobic amino acid and x denotes any amino acid; more particularly, such javelins may have the sequence hydrophobic-basic-hydrophobic-hydrophobic-hydrophobic; Ser/Thr-hydrophobic-hydrophobic-Ser/Thr; Ser/Thr-Ser/Thr-hydrophobic-hydrophobic-Ser/Thr-Ser/Thr; and Ser/Thr-Ser/Thr-hydrophobic-hydrophobic-hydrophobic. Alternatively, javelins may comprise heat shock binding peptides as described in Blond-Elguindi et al., 1993, Cell 75:717-728, including the consensus sequence hydrophobic-(Trp/X)-hydrophobic-X-hydrophobic-X-hydrophobic and the specific peptides His Trp Asp Phe Ala Trp Pro Trp (SEQ ID NO: 3) and Phe Trp Gly Leu Trp Pro Trp Glu (SEQ ID NO: 8); Auger et al., 1996, Nature Med. 2:306-310, including Gln Lys Arg Ala Ala (SEQ ID NO: 9) and Arg Arg Arg Ala Ala (SEQ ID NO: 10); Flynn et al., 1989, Science 245:385-390; Gragerov et al., 1994, J. Mol. Biol. 235:848-854; Terlecky et al., 1992, J. Biol. Chem. 26:9202-9202, Lys Phe Glu Arg Gln (SEQ ID NO: 11); and Nieland et al., 1996, Proc. Natl. Acad. Sci. U.S.A. 93:6135-6139, including the VSV8 peptide, Arg Gly Tyr Val Tyr Gln Gly Leu (SEQ ID NO: 12). In preferred embodiments, javelins of the invention may have a length of 4-50 amino acid residues, and more preferably 7-20 amino acid residues.

An epitope is defined as a molecule or region of a molecule against which an immune response is raised (cellular and/or antibody). In the case of peptidic epitopes, the epitope could constitute an MHC Class I binding peptide or an MHC Class II binding peptide ranging in size from 6 to 20 amino acids. Epitopes of the invention may be derived from virus proteins, bacterial proteins, protozoan proteins, fungal proteins, parasite derived proteins, intracellular pathogen derived proteins and proteins from diseased cells such as those derived from malignant tissue. According to the invention, an epitope need not be identified or characterized; its functional presence alone is required. Therefore, for example and not by way of limitation, a protein which is known to comprise a plurality of epitopes by virtue of the immune responses it induces may be javelinized according to the invention regardless of whether the peptide sequences or other characteristics of those epitopes are known.

A plurality of said epitopes may be comprised in a larger protein, which is in turn covalently linked to a javelin, optionally via a linker sequence. Epitopes, which may be comprised in larger molecules (such as larger peptides), may be covalently linked to javelin either in series (i.e., as part of a linear peptide molecule) or some epitopes may be linked to the javelin in parallel (i.e., via an amino acid side chain). Said plurality of epitopes may occur naturally in the same protein, or may occur in different proteins. For example, epitopes of proteins derived from a plurality of genetic variants of a virus may be linked to a javelin and incorporated into a heat shock protein complex of the invention.

In particular non-limiting embodiments of the invention, a protein antigen comprising a plurality of epitopes may have greater than 20 amino acids and may have one or more natural or heteroclitic MHC class I and/or MHC class II binding peptides, and may incorporate any additional immunogenic sequences including, but not limited to antibody recognition sites. Such a protein could constitute a naturally occurring protein or it could constitute a synthetic protein generated to contain one or more copies of MHC class I and/or MHC class II binding peptides.

The javelinization of epitopes can be carried out in a number of ways. For example, but not by way of limitation, a javelin can be chemically or photochemically crosslinked to one or more epitopes, or may be produced by genetic engineering techniques.

The molecule comprising the one or more epitopes is referred to herein as the “antigen”, such that the antigen is linked to a javelin.

Between a javelin and the antigen a linker molecule may be used. If this linker is peptidic, it could correspond to but is not limited to a 1-10 amino acid sequence. Such a linker could have the sequence but is not limited to GSG, GGSGG (SEQ ID NO: 13), GGPGG (SEQ ID NO: 14), SGPGS (SEQ ID NO: 15).

Antigens can be attached (by any of the methods described above) to one or more javelins. The javelin(s) can be placed at any point on a antigenic surface. In the case of peptidic antigens and a peptidic javelin, the javelin can be at the amino-terminus of the protein, the carboxyl terminus of the protein, or one or more javelins can be introduced at any point within the amino acid sequence of the protein antigen or any combination of the above.

Complexes between javelinized antigens and hsps and/or molecular chaperones can be generated by many methods. Javelinized antigens can be mixed with the hsps and/or molecular chaperones at molar ratios varying from but not limited to 0.01:1 to 100:1, although more preferably in molar ratios of 0.1:1 to 10:1. These mixtures are made in an aqueous solution that is buffered in the range between pH 4.5and pH 9 and more preferably in the range pH 5.5 to pH 8. The buffering compounds could include but are not limited to Tris base, phosphate based buffers, bicarbonate based buffers, succinate based buffers. The concentrations of these buffering compounds range from but is not limited to 1 mM to 500 mM, and more preferably range from 10 mM to 200 mM. Salts may also be added to the solution. These salts include but are not restricted to sodium chloride, potassium chloride, ammonium chloride, ammonium sulfate, magnesium chloride, magnesium acetate, potassium acetate, sodium acetate. The concentrations of these salts may fall in the range, but are not limited to, 1 mM to 500 mM, more preferably 20 mM to 200 mM. The formation of complexes between Javelinized antigens and hsps and/or molecular chaperones may also involve the addition of one or more salt to the complex formation solution. Other compound, not always referred to as salts may also be included in the complex formation solution. Such compounds may include but are not limited to adenosine 5′ diphosphate (ADP) and analogues thereof, adenosine 5′ triphosphate (ATP) and analogues thereof and DMSO. Such compounds may be added at concentrations ranging from but not limited to 0.001 mM to 500 mM, more preferably 0.1 mM to 100 mM.

An example of a complex formation solution is as follows, but is not in any way limiting:

hsp70 0.25 mg/ml

Javelinized antigen peptide at either 0.25 mg/ml in a buffer comprising:

25 mM Tris (Tris) pH 8.0

50 mM NaCl

5 mM MgCl₂

6.7 mM Acetate

1 mM ADP

0.26 mM KCl

0.518 mM Na₂HPO₄

0.173 mM KH₂PO₄

Final DMSO concentration 1%.

The complex formation reaction should then be incubated at a temperature ranging from, but not limited to 4° C. to 65° C., more preferably from 20° C. to 55° C. This incubation will be carried out for a time period ranging from, but not limited to 1 minute to 4 hours, more preferably from 20 minutes to 1 hour.

Immunizations of javelinized antigens bound to an hsp can be carried out in numerous ways. Immunization can be carried out using a single javelinized antigen bound to a heat shock protein or a plurality of javelinized antigens may be bound to a heat shock protein. Alternatively, a single javelinized antigen can be bound to numerous heat shock proteins or a plurality of javelinized antigens can be bound to a plurality of heat shock proteins. Additionally, one or more antigen can be variously javelinized and bound to a single or a plurality of heat shock proteins. Immunization can be carried out by methods including, but not limited to intradermal injection, subcutaneous injection, intraperitoneal injection and intramuscular injection. Immunization may also involve the treatment of patient derived antigen presenting cells with javelinized antigens bound to heat shock protein or molecular chaperone in vitro, followed by readministration of the antigen presenting cells into the patient.

Administration of javelinized antigen(s) bound to heat shock protein(s) may induce either a killer T cell response or a helper T cell response or an antibody response. More preferably, such an administration will induce both a helper T cell and killer T cell response or both a helper T cell and antibody response or both a killer T cell response and an antibody response. Even more preferably, such an administration will induce a killer T cell response, a helper T cell response and an antibody response.

Examples of javelinized antigens include but are not restricted to:

MHC Class I peptide antigen derived from ovalbumin containing one or two javelins, such as:

(SEQ ID NO:16)

SIINFEKLGSGHWDFAWPW;

(SEQ ID NO:17)

HWDFAWPWGSGSIINFEKL; and

(SEQ ID NO:18)

HWDFAWPWGSGSIINFEKLGSGHWDFAWPW

MHC Class II peptide antigen derived from ovalbumin containing one or two javelins, such as:

(SEQ ID NQ:19)

HWDFAWPWGSGTEWTSSNVMEERKIKV;

(SEQ ID NO:20)

TEWTSSNVMEERKIKVGSGHWDFAWPW; and

(SEQ ID NO:21)

HWDFAWPWGSGTEWTSSNVMEERKIKVGSGHWDFAWPW;

ID NO: 21);

MHC Class I peptide antigen derived from herpes simplex virus containing one or two javelins, such as:

| | |
|---|---|
| HWDFAWPWGSGSSIEFARL; | (SEQ ID NO:22) |
| SSIEFARLGSGHWDFAWPW; and | (SEQ ID NO:23) |
| HWDFAWPWGSGSSIEFARLGSGHWDFAWPW; | (SEQ ID NO:24) |

according to the present invention, a second epitope-containing peptide or a plurality of epitope-containing peptides, originating from the same herpes simplex protein or a different protein, may be linked to the javelin molecule(s) in peptides SEQ ID NOS: 22-24.

MHC class I mutant peptide antigen derived from gp100 containing one or two javelins, such as:

| | |
|---|---|
| HWDFAWPWGSGIMDQVPFSV; | (SEQ ID NO:25) |
| IMDQVPFSVGSGHWDFAWPW; and | (SEQ ID NO:26); |
| HWDFAWPWGSGIMDQVPFSVGSGHWDFAWPW; | (SEQ ID NO:27) |

according to the present invention, a second epitope-containing peptide or a plurality of epitope-containing peptides, originating from the same gp100 protein or a different protein, may be linked to the javelin molecule(s) in peptides SEQ ID NOS: 25-27.

and

Ovalbumin derived protein domain containing both an MHC class I and MHC class II epitope and one javelin with or without the linker GSG, such as:

(SEQ ID NO:28)

HWDFAWPWVTEQESKPVQMMYQIGLFRVASMASEKMKILELPFASGTMS
MLVLLPDEVSGLEQLESIINFEKLTEWTSSNVMEERKIKVYLPRMKMEE
KY;

(SEQ ID NO:29)

HWDFAWPWGSGVTEQESKPVQMMYQIGLFRVASMASEKMKILELPFASG
TMSMLVLLPDEVSGLEQLESIINFEKLTEWTSSNVMEERKIKVYLPRMK
MEEKY;

(SEQ ID NO:30)

VTEQESKPVQMMYQIGLFRVASMASEKMKILELPFASGTMSMLVLLPDE
VSGLEQLESIINFEKLTEWTSSNVMEERKIKVYLPRMKMEEKYHWDFAW
PW;

(SEQ ID NO:31)

VTEQESKPVQMMYQIGLFRVASMASEKMKILELPFASGTMSMLVLLPDE
VSGLEQLESIINFEKLTEWTSSNVMEERKIKVYLPRMKMEEKYGSGHWD
FAWPW;

(SEQ ID NO:32)

HWDFAWPWVTEQESKPVQMMYQIGLFRVASMASEKMKILELPFASGTMS
MLVLLPDEVSGLEQLESIINFEKLTEWTSSNVMEERKIKVYLPRMKMEE
KYHWDFAWPW; and

(SEQ ID NO:33)

HWDFAWPWGSGVTEQESKPVQMMYQIGLFRVASMASEKMKILELPFASG
TMSMLVLLPDEVSGLEQLESIINFEKLTEWTSSNVMEERKIKVYLPRMK
MEEKYHWDFAWPW;

where MHC class I epitopes are singly underlined, MHC class II epitopes are doubly underlined, the Javelin sequence is bolded and the linker is italicized.

### 6. EXAMPLE

Induction of a Protective Immune Response using a Javelinized Portion of Ovalbumin Containing both Class I and Class II Epitopes

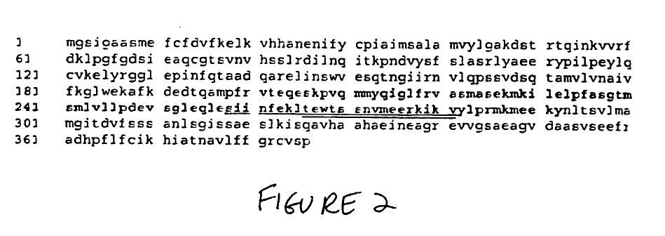

Good CTL responses have been generated when mice were immunized with a javelinized version of SIINFEKL (SEQ ID NO: 5; a peptide located from residues 257-264 in the ovalbumin protein) bound to hsp70 (see PCT/US 96/13363, and Moroi Y. et. al., (2000) Proc Natl. Acad. Sci. 97:3485-90). Thus, in the experiments described herein, the ovalbumin system was used to test the javelinization of large proteins. Ovalbumin is a ˜42.9 kDa protein with 386 amino acids that is secreted as a disulfide bonded molecule. Thus it is not very convenient to express this protein in E. coli. Furthermore, there have been no literature reports of soluble domains or fragments of ovalbumin that have been expressed. Therefore, in these experiments, a small 92 amino acid domain of ovalbumin (amino acids 200-291) was used, that contains the SIINFEKL (SEQ ID NO: 5) epitope from our Javelinization studies as well as an MHC class II peptide TEWTSSNVMEERKIKV (SEQ ID NO: 6) corresponding to residues 265-280 (Maecker H. T. et al., (1998) J. Immunol. 161:6532-6) The protein sequence of ovalbumin is shown in FIG. 2.

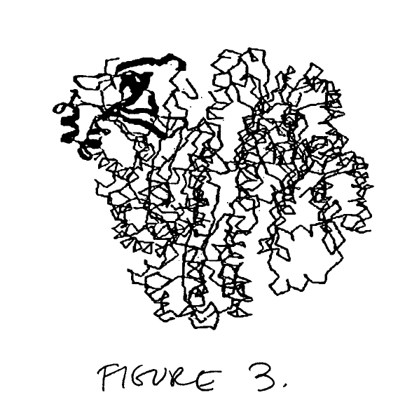

The bolded amino acids correspond to those of the domain 200-291. The MHC class I epitope, SIINFEKL (SEQ ID NO: 5), is underlined and the MHC class II epitope, TEWTSSNVMEERKIKV (SEQ ID NO: 6), is double underlined. This domain was chosen based on the fact that it contains both an MHC class I and II epitope, but also because structurally, the domain was considered to be relatively compact. The relatively compact nature of this domain may make it easier to work with (i.e. the domain would be relatively stable and be less susceptible to proteolysis or rapid aggregation even at low concentration). The structure of this domain in the context of the whole ovalbumin protein is shown in FIG. 3.

Manufacture of the Javelinized Ovalbumin Domains:

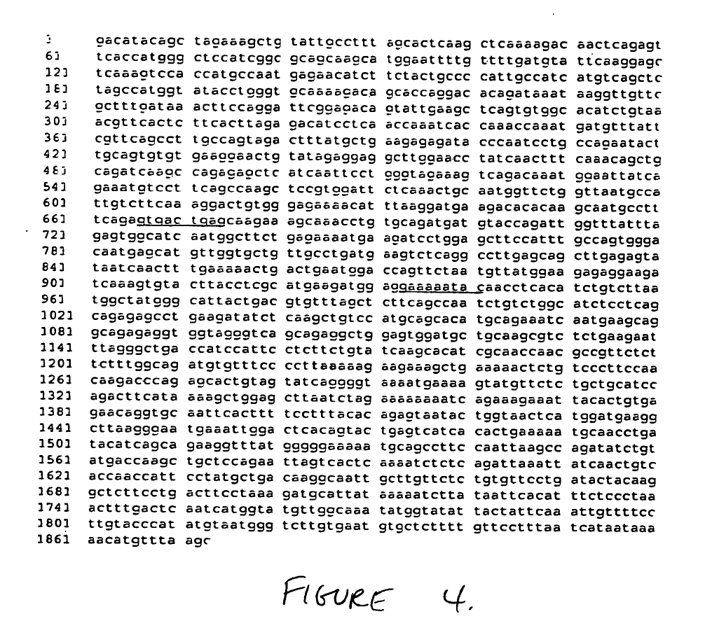

The nucleotide sequence of the ovalbumin mRNA is shown in FIG. 4. The ATG start codon is bolded as is the termination codon. Underlined are the 5′ and 3′ regions of the sequence that code for the OVA 200-291 domain. For your orientation, the first 9 bases of the 5′ sequence GTGACTGAG (SEQ ID NO: 34) codes for V-T-E and the last 9 bases of the 3′ sequence GAAAAATAC (SEQ ID NO: 35) codes for E-K-Y.

The following oligonucleotides were synthesized:

(SEQ ID NO:36)

5′ AACCCCATGTGACTGAGCAAGAAAGC 3′;

(SEQ ID NO:37)

5′ GCAAGGATCCTTAGTATTTTTCCTCC 3′;

(SEQ ID NO:38)

5′ GGAATTCCATATGCACTGGGACTTCGCGTGGCCGTGGGTGACTGAGC
AAGAAAGCAA 3′; and

(SEQ ID NO:39)

5′ GGAGGATCCTTACCACGGCCACGCGAAGTCCCAGTGGTATTTTTCCT
CCATCTTCATGCGA 3′.

These oligonucleotides have the following features:

1. Forward oligo coding for an NcoI site (CCATGG; SEQ ID NO: 40), the start codon (ATG) reading straight into the ovalbumin sequence (i.e. no bases coding for Javelin added);

2. Reverse oligo coding for a Bam HI site (GGATCC; SEQ ID NO: 41), a stop codon (TTA) immediately following ovalbumin sequence (remember this is a reverse complementary oligonucleotide);

3. Forward oligo coding for an NdeI site (CATATG; SEQ ID NO: 42), a start codon (ATG), 24 bases that code for the sequence of the Javelin (CACTGGGACTTCGCGTGGCCGTGG: (SEQ ID NO: 43) followed by ovalbumin sequence; and

4. Reverse oligo coding for a Bam HI site (GGATCC;SEQ ID NO: 44), a stop codon (TTA) which follows the sequence coding for the Javelin (CCACGGCCACGCGSAGTCCCAGTG(SEQ ID NO: 45). This immediately following ovalbumin sequence (remember this is a reverse complementary oligonucleotide).

Thus, these oligo's were used to generate, using the polymerase chain reaction, sequences corresponding to:

Ova 200-291 (no Javelinization)

Javelin-Ova 200-291 (One Javelin coding sequence at the 5′ end)

Ova200-291-Javelin (One Javelin coding sequence at the 3′ end)

Javelin-Ova200-291-Javelin (A Javelin coding sequence at both the 5′ and the 3′ end)

These sequences were subsequently cloned into pET vectors. The sequences corresponding to Ova 200-291 and Ova200-291-Javelin were cut with the restriction enzymes NcoI and Bam HI according to the enzyme manufacturers (New England Biolabs) instructions and cloned into pET28 (which had been similarly cut). The sequences corresponding to Jav-Ova and Jav-OVA-Jav were cut with NdeI and Bam HI according to the manufacturers instructions and cloned into pET27 (which had been cut with the same enzymes). The ligated vectors were transformed into the E.coli cell line HMS174. The resulting vectors were sequenced to confirm that no mutations had been introduced as a result of handling. All sequences were correct.

Expression and Purification of Ova200-291, Javelin-Ova200-291, Ova200-291-Javelin and Javelin-Ova200-291-Javelin:

This procedure is identical for the expression and purification of all of these proteins. A colony of HMS174 containing the vector of interest is picked and grown in LB medium supplemented with 30 g/ml of kanamycin. After overnight culture, the cells are spun out and resuspended in 5 ml of fresh LB medium. 1 ml of the resuspended cells is then used to innoculate a liter of LB medium which has been supplemented with 30 g/ml kanamycin. The cells are grown at 37° C. until the optical density at 600 nm is between 0.4-0.8 but preferably 0.6. At this point the culture is supplemented with IPTG at a final concentration of 1 mM. The culture is grown for an additional 3 hours and the cells spun down. Cell pellet can be stored frozen at this point.

The proteins are all found in inclusion bodies. Thus an inclusion body preparation is carried out. The cell lysis reagent Bugbuster™ sold by Novagen is used according to the manufacturers recommendation. Briefly, 5 ml of the Bugbuster™ reagent per gram of cell paste (pellet) is used to resuspend the pellet. 25 units of Benzonase (Novagen) is added for every ml of Bugbuster™ added. The cells are incubated at room temperature with gentle rotation for 10-20 minutes. The suspension is spun at 16000×g for 20 minutes at 4° C. and the pellet kept. The pellet is then resuspended in the same volume of Bugbuster™ as the original cell pellet was. Lysozyme is added to 200 g/ml, the sample vortexed and incubated for a further 5 minutes. 6 volumes of 1:10 fold diluted Bugbuster™ are then added and the suspension vortexed once again. The pellet is once again harvested by centrifugation (as above). The pellet is then resuspended in 20-30 ml of 1:10 fold diluted Bugbuster™ per liter of culture. The suspension is vortexed, spun and the pellet wash repeated 2 more times. The final pellet, containing purified inclusion bodies is dissolved in 50 mM Mops, pH6.5, 8M Urea, 0.1 mM DTT, 0.1 mM EDTA. The pellet goes into solution slowly over the course of about 1 hour (rotating at room temperature). The volume of urea is added such that the final concentration of protein is between 20-50 mg/ml. For the doubly Javelinized Ova200-291, the protein was not very soluble at pH 6.5 so KOH was added until the protein went back into solution. The pH after adjustment was approximately 10.

Immunization Studies:

An immunization study was initiated using the doubly Javelinized Ova200-291. The following solutions were set up:

100 μl TiterMax™ +100 μl buffer (50 mM NaCl, 20 mM Tris pH 8.0, 5 mM MgAc).

100 μl TiterMax™ +100 μl buffer containing 100 μg SIINFEKL (SEQ ID NO: 5) peptide.

1000 μl buffer containing 100 μg Javelin-Ova200-291-Javelin, 1 mM ADP.

1000 μl buffer containing 300 μg mouse hsp70, 100 μg Javelin-Ova200-291-Javelin, 1 mM ADP.

In samples C and D the buffer containing the other compounds listed was added very rapidly (by pipeting) onto the peptide (which is 8M urea). The TiterMax™ samples were vortexed for 30 minutes prior to immunization and the aqueous samples were incubated for a minimum of 30 minutes before immunization (The exact time may be more due to the time between formulating the solutions and actually administering the immunization).

10 mice were each immunized with one of the above mixtures. Thus 40 mice were immunized in total. For the TiterMax™ samples mice were immunized with 10 μl intradermally while for the aqueous preparations, each mouse was immunized with 50 μl intradermally. Thus mice in the following groups received the following moles of active ingredients:

Group A: no peptide, no hsp7O

Group B: 5000 pmoles SIINFEKL (SEQ ID NO:S), no hsp70

Group C: 365 pmoles Javelin-OVA200-291-Javelin, no hsp70

Group D: 365 pmoles Javelin-OVA200-291-Javelin, 214 pmoles mouse hsp70

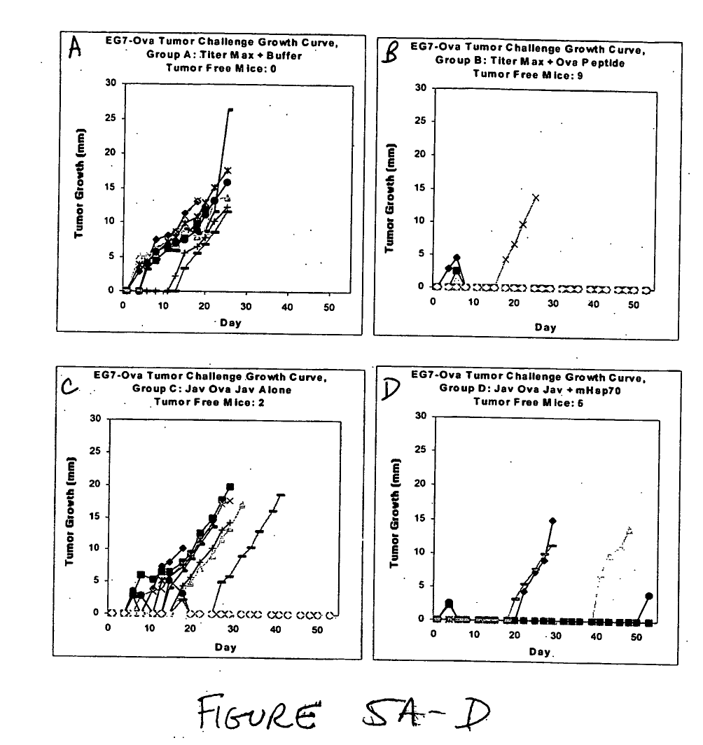

After 7 days, the mice were each challenged with 1×10⁶ EG7 cells intradermally. EG7 is a tumor cell line (derived from EL4) that has been stably transfected with the ovalbumin gene. Thus, if our immunizations result in the development of good immune responses to ovalbumin, the mice should be able to clear the tumor. Tumor growth measurements are then carried out at regular 2 day intervals. The responses observed for this experiment can be seen in FIG. 5 (note: two mice from the Hsp70: Javelin-Ova200-291-Javelin group (group D) died during anesthesia, hence only 8 mice are represented in the graph). These data clearly indicate that while all the mice immunized with TiterMax™ +buffer (group A) succumbed to the tumors, the mice immunized with Javelin-OVA200-291-Javelin bound to hsp70 (group D) had either no tumor (6/8) or had prolonged times to onset of disease (2/8). Mice immunized with TiterMax™ +SIINFEKL (SEQ ID NO: 5) peptide (group B) also, as expected, resisted disease well, while mice immunized only with Javelin-Ova200-291-Javelin (group C) had slightly increased survival times, but the response was not as significant in the group with hsp70. This clearly indicates the potential of this therapeutic approach.

### 7. EXAMPLE

Expression of Peptides or Proteins in Mammalian Cells

The foregoing example section utlized a bacterial system to express a protein for use according to the invention. However, typically expression of mammalian proteins in bacterial expression systems can be problematic in view of the lack of various modification systems (e.g. for glycosylation) that are lacking in bacterial cells. It therefore may be preferable to use a mammalian system to express a peptide or protein for use according to the invention. The following is a specific, non-limiting example of how a mammalian expression system may be used to produce a javelinized ovalbumin protein.

To create an expressible vector encoding the Javelinized and non-Javelinized Ova domains above in a mammalian cell, a Hind III may be created at the 5′ end of the Nco I restriction sites of the Nco I-BamH I fragments encoding Ova 200-291 and Ova200-291-Jav by PCR cloning from the bacterial expression system described in the section above. Replacement with a Hind III site will facilitate cloning into most mammalian expression vector systems such as pCDNA3.1 (In-Vitrogen) using standard techniques in Molecular Biology.(Molecular Cloning, Sambrook). Similarly, a Hind III site may be added to 5′end of the Nde I restriction sites of the Nde I-Bam-HI fragments encoding Jav-Ova and Jav-Ova-Jav.

5′ primers which may be used are:

^{5′}CCCAAGCTTGGGCCATGGTGACTGAGCAAGAAAGC^{3′} (SEQ ID NO: 46; addition of Hind III restriction site at the 5′ end of the Nco I site of primer 1 (supra)

^{5′}CCCAAGCTTGGGCATATGCACTGGGACTTCGCGTGGCCGTGGGTGACTG AGCAAGAAAGCAA^{3′}(SEQ ID NO; 47; addition of Hind III restriction site at the 5′ end of the Nde I site of primer 3 (supra)

Plasmids encoding Ova200-291, Javelin-Ova200-291, Ova200-291-Javelin and Javelin-Ova200-291-Javelin may each be transfected into a mammalian cell line such as CHO cells. Cells may be transfected with 2 μg mammalian expression vector encoding Ova 200-291, Ova200-291-Jav, Jav-Ova and Jav-Ova-Jav using methods well known in the art such as Lipofectamine (Gibco BRL) according to the manufacturer's directions. Briefly, the expression vector and 6 μL of Lipofectamine may be diluted separately in 100 μL serum-free medium (OPTI-MEM I Reduced Serum Medium, Gibco BRL). The two solutions may then be mixed and incubated at room temperature for 45 minutes to allow formation of DNA-liposome complexes. 800 μL OPTI-MEM may be added to the complexes, mixed, and overlaid onto rinsed cells. After a 6-hour incubation at 37° C., 1 mL growth medium containing 20% FCS may be added. Fresh medium may be added to the cells 24 hours post-transfection.

Stable clones may be selected by adding 800 μg/mL Geneticin (Gibco BRL) to the cells 72 hour later. The selection medium may be changed every 3 days. Colonies of stably transfected cells may be expected to be seen after 10-14 days. Expression of the desired javelinized Ova proteins may be assayed for by radiolabeling. Newly synthesized Ova may be detectable by immunoprecipitation and gel electrophoresis. Alternately, expression of the Ova producing clones may be confirmed by fluorescent-labeling of the permeabilized fixed cells and analyzed by fluorescent activated cell sorter (FACS analysis). Since the Ova protein is a secretable protein, Brefeldin A (BFA) may be added to the cells to prevent transport and hence secretion of the Ova protein prior to fluorescent labeling.

Analogous procedures may be used to express other peptides or proteins for use according to the invention.

### 8. EXAMPLE

Determination of Immunogenicity of HSP/Javelin—Protein Complexes

The following is a protocol which may be used to determine the immunogenicity of hsp/javelinized protein complexes (HSP/Jav-Protein).

Purified javelinized protein may be complexed with recombinant hsp in vitro at molar ratios of 10:1 to 500:1, and preferably at molar ratio of 10:1 to 100:1. The mixture may be incubated in a salt containing buffer in the range of pH4.5 and pH9 and more preferably in the range of pH6 to pH8. Examples of buffering systems include Tris-based buffer, phosphate-based buffer, citrate based buffer, succinate based buffer, bicarbonate based buffer and Hepes based buffer. The concentrations may range from 1 to 500 mM and preferably range from 10 to 100 mM. The mixture may be incubated at 25° C. for 20-120 minutes. The resulting HSP/Jav-Protein complexes may be assayed for immunogenicity using cytotoxic T cell assay.

Mice may be immunized intra-dermally once with 10-50 μL of HSP/Jav-Protein complex. Ten days after immunization, the spleens may be removed and the lymphocytes may be cultured with restimulation in vitro by the addition of whole protein or taansfected cells expressing the javelinized protein. If a pathogen protein is javelinized, the stimulation cells can be inactivated pathogen infected cells. Inactivation can be achieved by irradiation at 3000 rads or by treatment with mitomycin C.

Cytotoxicity of spleen cells from vaccinated mice may be assayed with either protein-pulsed cells or target cells expressing the protein. CTL may be generated by culturing in vivo immunized spleen cells for 5-6 days at a concentration of 1-10×10⁶ cells/mL in RPMI medium containing 10% FCS, penicillin-streptomycin and 2 mM glutamine, together with 1-5×10⁴ gamma irradiated stimulator cells/mL. Target cells may be prepared by culturing cells for 1 h in the presence of 200 mCi ⁵¹Cr (sodium chromate)/mL (NEN) in Tris-phosphate buffer, pH 7.4 at 37° C. After washing, 10^{4 51}Cr-labeled target cells may be mixed with effector lymphocytes to yield several different Effector/Target (E/T) ratio and incubated for 4 h. The cells may be pelleted by centrifugation at 200×g for 5 minutes and the amount of ⁵¹Cr released into the supernatant determined using a gamma counter. Percent specific lysis may be calculated as 100%×[(cpm released by CTL−cpm spontaneous release)/(cpm maximal release−cpm spontaneous release)]. Maximal release may be determined by addition of 1% Triton X-100. Spontaneous release by target cells in the absence of effector cells is typically less than 25% of themaximal release.

Various publications are cited herein, the contents of which are hereby incorporated by reference in their entireties.

What is claimed is:

1. An antigenic complex comprising a plurality of epitopes, non-covalently joined to a heat shock protein by a tethering molecule having affinity for the heat shock protein under physiologic conditions, wherein the epitopes are covalently joined to the tethering molecule and wherein one epitope is a Class I epitope and the other epitope is a Class II epitope.

2. An antigenic complex comprising a plurality of epitopes, non-covalently joined to a heat shock protein by a tethering molecule having affinity for the heat shock protein, wherein the epitopes are covalently joined to the tethering molecule and wherein the epitopes are derived from more than one antigenic protein.

3. An antigenic complex comprising one or more epitope, as comprised in an antigenic peptide, non-covalently joined to a heat shock protein by a plurality of tethering molecules, wherein the epitope or epitopes are covalently joined to the tethering molecule.


SEQUENCE LISTING (47 sequences)

SEQ ID NO: 1 - protein, 4 aa, Homo sapiens
KDEL

SEQ ID NO: 2 - protein, 4 aa, Plasmodium falciparum
NANP

SEQ ID NO: 3 - protein, 8 aa, Aritifical Sequence
  insert in M13 coliphage
HWDFAWPW

SEQ ID NO: 4 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 5 - protein, 8 aa, Gallus gallus
SIINFEKL

SEQ ID NO: 6 - protein, 16 aa, Gallus gallus
TEWTSSNVMEERKIKV

SEQ ID NO: 7 - DNA, 1873 bp, Gallus gallus
gacatacagctagaaagctgtattgcctttagcactcaagctcaaaagacaactcagagttcaccatgggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacagataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaaaaagaagaaagctgaaaaactctgtcccttccaacaagacccagagcactgtagtatcaggggtaaaatgaaaagtatgttctctgctgcatccagacttcataaaagctggagcttaatctagaaaaaaaatcagaaagaaattacactgtgagaacaggtgcaattcacttttcctttacacagagtaatactggtaactcatggatgaaggcttaagggaatgaaattggactcacagtactgagtcatcacactgaaaaatgcaacctgatacatcagcagaaggtttatgggggaaaaatgcagccttccaattaagccagatatctgtatgaccaagctgctccagaattagtcactcaaaatctctcagattaaattatcaactgtcaccaaccattcctatgctgacaaggcaattgcttgttctctgtgttcctgatactacaaggctcttcctgacttcctaaagatgcattataaaaatcttataattcacatttctccctaaactttgactcaatcatggtatgttggcaaatatggtatattactattcaaattgttttccttgtacccatatgtaatgggtcttgtgaatgtgctcttttgttcctttaatcataataaaaacatgtttaagc

SEQ ID NO: 8 - protein, 8 aa, Aritifical Sequence
  insert in M13 coliphage
FWGLWPWE

SEQ ID NO: 9 - protein, 5 aa, Aritifical Sequence
  insert in M13 coliphage
QKRAA

SEQ ID NO: 10 - protein, 5 aa, Aritifical Sequence
  insert in M13 coliphage
RRRAA

SEQ ID NO: 11 - protein, 5 aa, vesicular stomatitis virus
KFERQ

SEQ ID NO: 12 - protein, 8 aa, vesicular stomatitis virus
RGYVYQGL

SEQ ID NO: 13 - protein, 5 aa, Aritifical Sequence
  synthetic peptide linker
GGSGG

SEQ ID NO: 14 - protein, 5 aa, Aritifical Sequence
  synthetic peptide linker
GGPGG

SEQ ID NO: 15 - protein, 5 aa, Aritifical Sequence
  synthetic peptide linker
SGPGS

SEQ ID NO: 16 - protein, 19 aa, Aritifical Sequence
  javelinized peptide from Gallus gallus ovalbumin
SIINFEKLGSGHWDFAWPW

SEQ ID NO: 17 - protein, 19 aa, Aritifical Sequence
  javelinized peptide of Gallus gallus ovalbumin
HWDFAWPWGSGSIINFEKL

SEQ ID NO: 18 - protein, 30 aa, Aritifical Sequence
  javelinized peptide of Gallus gallus ovalbumin
HWDFAWPWGSGSIINFEKLGSGHWDFAWPW

SEQ ID NO: 19 - protein, 27 aa, Aritifical Sequence
  javelinized peptide of Gallus gallus ovalbumin
HWDFAWPWGSGTEWTSSNVMEERKIKV

SEQ ID NO: 20 - protein, 27 aa, Aritifical Sequence
  javelinized peptide of Gallus gallus ovalbumin
TEWTSSNVMEERKIKVGSGHWDFAWPW

SEQ ID NO: 21 - protein, 38 aa, Aritifical Sequence
  javelinized peptide of Gallus gallus ovalbumin
HWDFAWPWGSGTEWTSSNVMEERKIKVGSGHWDFAWPW

SEQ ID NO: 22 - protein, 19 aa, Aritifical Sequence
  javelinized peptide of Herpes Simplex virus
HWDFAWPWGSGSSIEFARL

SEQ ID NO: 23 - protein, 19 aa, Aritifical Sequence
  javelinized peptide of Herpes Simplex virus
SSIEFARLGSGHWDFAWPW

SEQ ID NO: 24 - protein, 30 aa, Aritifical Sequence
  javelinized peptide of Herpes Simplex virus
HWDFAWPWGSGSSIEFARLGSGHWDFAWPW

SEQ ID NO: 25 - protein, 20 aa, Aritifical Sequence
  javelinized peptide of gp100 human melanoma antigen
HWDFAWPWGSGIMDQVPFSV

SEQ ID NO: 26 - protein, 20 aa, Aritifical Sequence
  javelinized peptide of human gp100 melanoma antigen
IMDQVPFSVGSGHWDFAWPW

SEQ ID NO: 27 - protein, 31 aa, Aritifical Sequence
  javelinized peptide of gp100 melanoma antigen
HWDFAWPWGSGIMDQVPFSVGSGHWDFAWPW

SEQ ID NO: 28 - protein, 100 aa, Aritifical Sequence
  javelinized peptide of Gallus gallus ovalbumin
HWDFAWPWVTEQESKPVQMMYQIGLFRVASMASEKMKILELPFASGTMSMLVLLPDEVSGLEQLESIINFEKLTEWTSSNVMEERKIKVYLPRMKMEEKY

SEQ ID NO: 29 - protein, 103 aa, Aritifical Sequence
  javelinized peptide of Gallus gallus ovalbumin
HWDFAWPWGSGVTEQESKPVQMMYQIGLFRVASMASEKMKILELPFASGTMSMLVLLPDEVSGLEQLESIINFEKLTEWTSSNVMEERKIKVYLPRMKMEEKY

SEQ ID NO: 30 - protein, 100 aa, Aritifical Sequence
  javelinized peptide of Gallus gallus ovalbumin
VTEQESKPVQMMYQIGLFRVASMASEKMKILELPFASGTMSMLVLLPDEVSGLEQLESIINFEKLTEWTSSNVMEERKIKVYLPRMKMEEKYHWDFAWPW

SEQ ID NO: 31 - protein, 103 aa, Aritifical Sequence
  javelinized peptide of Gallus gallus ovalbumin
VTEQESKPVQMMYQIGLFRVASMASEKMKILELPFASGTMSMLVLLPDEVSGLEQLESIINFEKLTEWTSSNVMEERKIKVYLPRMKMEEKYGSGHWDFAWPW

SEQ ID NO: 32 - protein, 108 aa, Aritifical Sequence
  javelinized peptide of Gallus gallus ovalbumin
HWDFAWPWVTEQESKPVQMMYQIGLFRVASMASEKMKILELPFASGTMSMLVLLPDEVSGLEQLESIINFEKLTEWTSSNVMEERKIKVYLPRMKMEEKYHWDFAWPW

SEQ ID NO: 33 - protein, 111 aa, Aritifical Sequence
  javelinized peptide of Gallus gallus ovalbumin
HWDFAWPWGSGVTEQESKPVQMMYQIGLFRVASMASEKMKILELPFASGTMSMLVLLPDEVSGLEQLESIINFEKLTEWTSSNVMEERKIKVYLPRMKMEEKYHWDFAWPW

SEQ ID NO: 34 - DNA, 9 bp, Artificial Sequence
  primer designed based on Gallus gallus ovalbumin cDNA
gtgactgag

SEQ ID NO: 35 - DNA, 9 bp, Artificial Sequence
  primer based on Gallus gallus ovalbumin cDNA
gaaaaatac

SEQ ID NO: 36 - DNA, 27 bp, Artificial Sequence
  primer based on Gallus gallus ovalbumin cDNA
aaccccatggtgactgagcaagaaagc

SEQ ID NO: 37 - DNA, 26 bp, Artificial Sequence
  primer based on Gallus gallus ovalbumin cDNA
gcaaggatccttagtatttttcctcc

SEQ ID NO: 38 - DNA, 57 bp, Artificial Sequence
  primer based on Gallus gallus ovalbumin cDNA
ggaattccatatgcactgggacttcgcgtggccgtgggtgactgagcaagaaagcaa

SEQ ID NO: 39 - DNA, 61 bp, Artificial Sequence
  primer based on Gallus gallus ovalbumin cDNA
ggaggatccttaccacggccacgcgaagtcccagtggtatttttcctccatcttcatgcga

SEQ ID NO: 40 - DNA, 6 bp, Artificial Sequence
  primer based on Gallus gallus ovalbumin cDNA
ccatgg

SEQ ID NO: 41 - DNA, 6 bp, Artificial Sequence
  primer based on Gallus gallus ovalbumin cDNA
ggatcc

SEQ ID NO: 42 - DNA, 6 bp, Artificial Sequence
  primer based on Gallus gallus ovalbumin cDNA
catatg

SEQ ID NO: 43 - DNA, 24 bp, Artificial Sequence
  artificial sequence in M13 coliphage
cactgggacttcgcgtggccgtgg

SEQ ID NO: 44 - DNA, 6 bp, Artificial Sequence
  primer based on Gallus gallus ovalbumin cDNA
ggatcc

SEQ ID NO: 45 - DNA, 24 bp, Artificial Sequence
  artificial sequence in M13 coliphage
ccacggccacgcgaagtcccagtg

SEQ ID NO: 46 - DNA, 35 bp, Artificial Sequence
  primer based on Gallus gallus ovalbumin cDNA
cccaagcttgggccatggtgactgagcaagaaagc

SEQ ID NO: 47 - DNA, 62 bp, Artificial Sequence
  primer based on Gallus gallus ovalbumin cDNA
cccaagcttgggcatatgcactgggacttcgcgtggccgtgggtgactgagcaagaaagcaa